(19) United States
(12) Patent Application Publication
(10) Pub. No.: US 2022/0073847 A1
(43) Pub. Date: Mar. 10, 2022

(54) SUBSTRATE DECOMPOSITION FOR BIOGAS PLANTS IN A MIXING AND COMBI-HYDROLYSIS TANK

(71) Applicant: PLANUNGSBÜRO ROSSOW GESELLSCHAFT FÜR ERNEUERBARE ENERGIEN MBH, Neubrandenburg (DE)

(72) Inventor: Norbert ROSSOW, Penzlin (DE)

(21) Appl. No.: 14/649,812

(22) PCT Filed: Dec. 9, 2013
(86) PCT No.: PCT/EP2013/075993

§ 371 Date: May 2, 2016

(30) Foreign Application Priority Data
Dec. 7, 2012 (DE) 10 2012 222 589.7

(51) Int. Cl. C12M 1/107; C12M 1/00; C12M 1/06; C12M 1/34; C12P 5/02

(52) U.S. Cl. CPC C12M 21/04; C12M 23/02; C12M 23/34; C12M 23/40; C12P 5/023; C12M 29/08; C12M 29/26; C12M 41/44; C12M 45/07; C12M 27/02

(57) ABSTRACT

fermelnters or other processes for the treatment or conversion of organic substances, and also for improving viscosity, in which specifically required, technological or biological procedures are combined in one mixing and combi-hydrolysis tank. The device has one or more descending walls for separating the gas chamber in the top part of the tank, one descending wall of which separates the gas chamber of the tank opening from the other tank, and optionally a further descending wall, the lower end of which is permanently located above the lowest fluid level and which separates the gas chamber of the pump chamber from the other tank. The device optionally has an ultrasonic module for treating a recirculated material from an advanced fermentation stage or from the device according to claim 1, and also a gas supply system in the lower zones of the feed and/or pump chamber for hydrogenous gases, produced in the process or supplied externally, for stimulating methane formation by means of hydrogen-oxidising archaea.

### BACKGROUND OF THE INVENTION

The invention relates to a method and a device for optimizing the acceptance, conditioning, hydrolysis, disintegration and methane enrichment of organic matter for feeding biogas fermenters or other processes for the treatment or conversion of organic substances, and also for improving viscosity, in which specifically required, technological or biological procedures are combined in one mixing and combi-hydrolysis tank. Optionally, ultrasonic treatment can be conducted additionally.

The limitations and scarcity of conventional energy sources requires efforts to use alternatives. In this context, renewable energy sources are increasingly important. Among these alternatives is also the generation of biogas through fermentation of organic biomass and animal excrement in biogas plants and their further energy exploitation. Since the provision and operation of these facilities cannot usually be achieved so as to cover costs without public funding, and because awareness with regard to environmental issues is increasing, the problem is the focus of social discourse. Steps towards the technical and economic optimization of such systems, and measures to achieve resource-saving and environmentally sound consumption, are therefore warranted.

Processes for the fermentation of excrement from animal sources, organic substrates or specially cultivated raw materials in a fermenter or reactor to subsequently generate biogas for the purpose of energy recovery have been in use for a considerable time already. The document DE 31 00 324 describes an apparatus for the conversion of biomass into energy by means of a gastight anaerobic fermentation chamber that is filled with biomass through a feed line and which is provided with a gas outlet.

Document DE 195 38 579 also describes a plant for the generation of biogas from organic matter in a gas-tight reactor with a slurry inlet and outlet as well as biogas extraction, an agitator for mixing and a device for substrate heating.

Likewise, in document DE102005 054 323 relates to a fermenter used for the generation of biogas from organic matter, characterized by a fermentation chamber for the intake of fermentation material, provided with a filling device, gas tank with gas extraction unit, agitator unit, stabilizing chamber with overflow edge, pump unit, and so forth.

The document cited last also refers to the different phases of the fermenting process. In the first step, the hydrolysis phase, carbohydrates are decomposed to simple sugars, proteins to amino acids and fats to fatty acids. An acid formation phase follows thereupon, to produce organic acids and lower alcohols, and a phase of acetic acid formation. Only thereafter does the methane formation (methanogenesis) phase follow. The distinction between these phases is technically and biologically of great importance in the practical implementation of biogas technology as each specific strain of microorganism has its own activity, and—as concerns the compatibility and optimal conditions of their effect—requires or prefers quite special environments in terms of acid status, temperature or aerobic sensitivity.

In the process of the further evolution of biogas plants, they have therefore transitioned to the spatial separation of the hydrolysis phase for the conversion of the material substrate from the other phases in order to increase the gas yield and stabilize its production. Accordingly, document WO 2011 138 426 provides a process and a system for two or multi-stage biogas generation in a hydrolysis and a methane stage.

Initially, mixing tanks were used for the initiation of hydrolysis. Consequently, these were more than just storage tanks for the substrate. There excrement such as slurry and manure, renewable raw materials, co-fermentates, possibly bio-waste as well as industrial and agricultural waste materials were effectively prepared for optimal digestion in the fermenter and, methane formation was already initiated to a certain extent.

Since the mixing tanks were usually open, energy losses were incurred and foremost, there was significant exposure to odour or gas emissions. The further development therefore moved towards the use of separate hydrolysis tanks upstream from the fermenters. In principle, these are—closed, gas-tight, provided with an agitator and, more rarely, with a heater—configured in the same way as a fermenter.

Before the substrate can reach the hydrolysis tank, it has to be prepared. The present state of the art frequently uses feed-dosing units for this purpose. These can often store a daily ration of the substrate. If applicable, certain substrates, e.g. solid manure, have to be additionally conditioned beforehand with a shredder, extruder or hammer mill, so as to avoid problems in further transport and homogenization later on.

The dosing units are equipped with augers, push rods or chain haulage units and they can thereby fulfil the functions of uptake, storing, loosening, crushing, mixing, portioning or dosing for the further processing. From the discharge point of the dosing unit, the fermenting material can be transported further on screw conveyors to the hydrolysis tank.

At the present time, this task, however, is also resolved occasionally by liquefaction of the substrate through re-circulation of the fermenter contents by means of pumps, which is a process that is still in development. By means of barrier and control apparatuses, the transport of the liquid media is possible in all desired quantities and directions.

In document WO 2006/124781, a multi-stage fermenter is described with at least three connected chambers, through which, one after the other, an organic waste substrate passes (e.g. sludge from wastewater cleaning) in an upstream and downstream flow sequence. The hydrolysis stage is part of the fermenter, while any mechanical pre-treatment of the organic substrate takes place outside of the fermenter.

In document U.S. Pat. No. 3,054,602, a segmented fermenter—similar to the one mentioned above—is described, which however is used for aerobic bacterial wastewater treatment without the formation of usable biogas. In an upstream and downstream flow sequence, hydrolysis takes place in an oxygen atmosphere, followed by further ventilated (and thus, aerobic) decomposition processes.

Document DE 3810250, as in the document mentioned above, describes a process and apparatus for the treatment of fluid substrates with a high organic load. Here, under anaerobic conditions, hydrolysis and methane formation take place in an upstream and downstream flow in a common fermenter, divided by a series of vertical lowering and stationary walls.

The two-stage anaerobic process for the generation of biogas from biomass as described in document WO 2008/099227 likewise uses an apparatus with a series of vertical lowering and stationary walls for separation into three (or more) chambers. In this case, as well, a sequence of upstream and downstream flows of the fluid is generated. This behaviour of the fluid is supported by injecting biogas. An explicit hydrolysis chamber is not mentioned as being part of the invention.

In the examined and published application DE 1301599, an apparatus is described for stirring, homogenization and discharge of viscous media, characterized in that special cutting pumps are used in a pump sump. They are used, in particular, to prepare slurry and solid manure in such a way that these substances can be extracted from tanks.

The fermenter, as published in document DE 102009021015, serves for the generation of biogas from biomass, functioning according to the principle of solid matter methanization. The percolator process described therein dispenses with a clearly defined device for hydrolysis. A separated fluid is sprayed on the solid matter but it is not stirred.

The drying fermenter described in the disclosure document DE 102006047828 is also operated in a percolation process and it is not equipped with any installations or agitators. The biomass is discharged via an aslant plane in the direction of the outlet. A separate chamber for a hydrolysis process is not part of this procedure.

A variant of an aerobic hydrolysis apparatus as a part of an anaerobic fermentation procedure is also presented in published patent application US 2010/0032370. The open hydrolysis chamber is structurally connected directly with the fermenter chamber so that the substrate mixture in the hydrolysis chamber reaches directly into the fermenter chamber by overflowing over a lowering wall. The generated hydrolysis gas dissipates unused from the chamber.

For biological wastewater treatment, a procedure using spatially separated aerobic/anaerobic zones is described in the patent specification U.S. Pat. No. 4,325,823. While only mixing takes place in the first chamber of the apparatus, the decomposition of the organic load of the wastewater takes place in the second chamber. Solid matter cannot be integrated. The targeted formation or use of gas does not take place.

Document DE 102010010294 describes a procedure for anaerobic fermentation of a flowing substrate. The described apparatus includes a plurality of sequential stationary and lowering walls between the inlet and outlet. These are designed as flow panels causing a change between upstream and downstream flow, and consequently form different reaction zones. A mixing and preparation zone is not provided and neither is any explicitly defined hydrolysis chamber.

The invention has the basic purpose of providing a significant simplification of the process of acceptance, i.e., preparation of organic matter to make it acceptable for further processing with the ultimate objective of producing biogas, conditioning and hydrolysis of organic matter for feeding biogas fermenters, as well as combining them by completing this process in just one tank instead of multiple units, as has been the case until now.

### SUMMARY OF THE INVENTION

According to the invention, the specific necessary technical or biological as well as physical procedures, which have been designed as separate from each other until now, have been combined in one mixing, feeding, dosing, disintegration and hydrolysis combi-tank, whereby the conversion of the energy potential of the input materials used is optimally tapped. Expenditures are thereby reduced to the minimum necessary to make the operation of biogas plants more cost-efficient and, additionally, to make ongoing operation more effective in terms of business management.

For this purpose, a preferably elongated, horizontal tank is used. It consists of concrete, steel, plastic or other suitable materials or material mixtures. For the interior walls, acid resistance must be ensured.

The tank can be recessed. If it is at ground level, it is provided with a ramp on one side or it must be fed by means of lifting-loading technology.

The invention will be described in greater detail with reference to the drawings.

### BRIEF DESCRIPTION OF THE DRAWINGS

FIG. 1 shows a layout of a tank according to the invention in longitudinal cross-section;

FIG. 2 is a cross-sectional view of a further embodiment of the invention; and

FIG. 3 is a top plan view of the FIG. 2 embodiment.

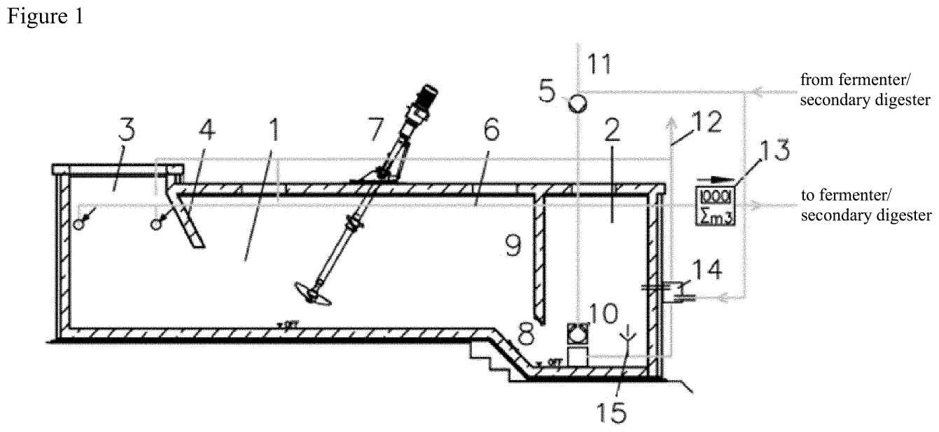

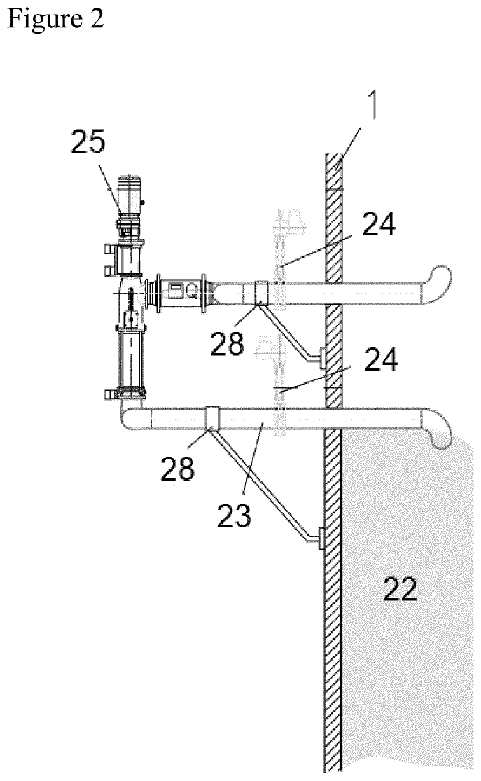

The tank is essentially divided, as shown in FIG. 1 and FIG. 2 into a biomass-feeding chamber (1) and a pump chamber (2). Both chambers are insulated and heatable. The upper end of the biomass-feeding chamber has an elongated, odour-inhibiting or gas-tight sealable opening (3) adjacent a drop wall (4).

The biomass is fed through this opening by material handling equipment, e.g. wheel loaders or dump trucks empty this material into the tank opening from a runway next to the tank or from the ramp. If necessary, the tank can also be filled via an injection shaft. The biomass feeding chamber (1) is thereby filled with fresh substrate. The hydrolysis phase begins and is sustainably supported in that biologically-active fluid can be extracted by means of a feed pump (5), preferably from the fermenter or, instead, from an external source, whereby it is applied on the surface of the fresh material from an injection system (6) installed in the chamber. The injection fluid moistens the substrate in a jet-like method. Material already moistened is microbiologically and enzymatically active, and it continues to be soaked repeatedly with fresh fluid medium, whereby the hydrolysis processes are optimised. In the process, the fluid level rises and all empty spaces around and within the fed-in substrate are filled up. From a programmed fill level, it can be transitioned to additionally treating the substrate, in that the mixing is sustained through periodic use of an agitator (7) and, successively, homogenization. When a maximum value is reached, the fluid supply is stopped.

The pump chamber is connected with the feed chamber through a slanted floor (8) or a recess where the overflow of the mashed substrate is carried. An optionally height-adjustable drop wall (9) ensures the retention of the solid mass and, foremost, effects a separation of the two chambers on the gas side. On the floor of the pump chamber, a cutting pump (10) or a line leading to this pump is installed; the related pump sump is deeper than the remaining tank floor and the submersion chamber is permanently filled with fluid.

If the material having been mixed in the tank, which is already largely homogenous, has reached the required maturity state with regard to hydrolysis, the substrate is aspirated in batches into the pump chamber by means of this pump and, through the cutting effect of the pump, it is further refined in its consistency, homogenized and then fed into the fermenter for the further fermenting stages. Otherwise, the material from the pump chamber can initially be once or repeatedly circulated in the feeding chamber and then fed again for comminution and homogenization in the pump chamber.

To eliminate blockages and any unwanted substrate lumps or encrustations encountered, and to generally support the transport of the deposited material from the biomass-feeding chamber to the pump chamber, the in-feed pump can flush and clear the biomass-feeding chamber with recirculation fluid via a flushing connection (11) under the required pressure.

Overall, the pump chamber serves for the storage of the further processable substrate, separation of the freshly fed-in and still-floating substrates, and continuous feed to the downstream plant according to need, and provision of a reservoir for hydrolysis bacteria.

The biogas created in the two chambers—which are optionally separated on the gas side or, instead, connected according to need—is fed into the central gas grid of the biogas plant through extraction outlets (12), either by means of the natural gas pressure from the gas formation phase, or it is specifically fed into the lower fluid zone of the feeding chamber or, as needed, into the pump chamber by means of a ventilator.

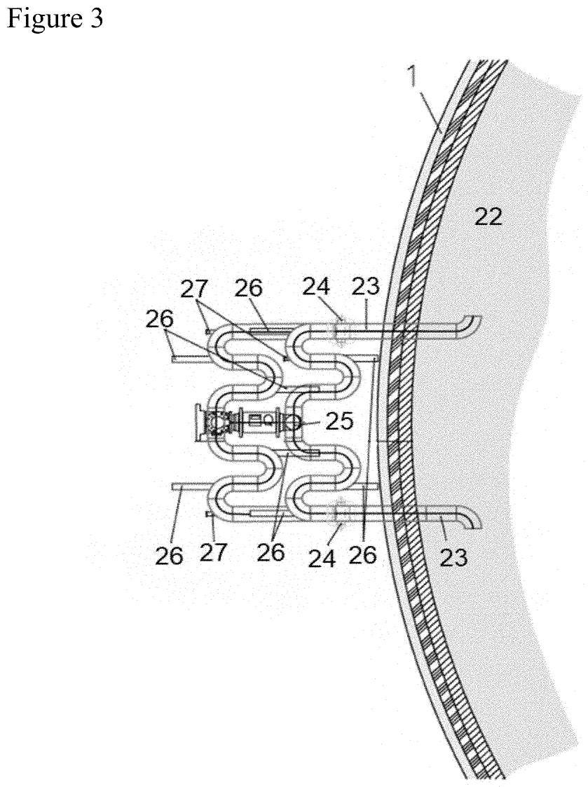

Previously, control of the gas production in the fermenter used relatively precise but indirect dosing of the biomass monitored by means of a weighing system through feeding tanks/devices, but is now provided in the present invention as direct control of the hydrolysis material being added in balance with present gas production from the biogas plant, which is facilitated by means of volume flow-measuring devices (13). Reference number 15 indicates a schematic representation of hydrolysis and hydrogen gas. An ultrasonic module 14 is shown schematically in FIG. 1 and in detail in FIGS. 2 and 3.

The process and de vice according to the invention enable a combined feeding, mixing, dosing and hydrolysis of biomass in one single tank for use in a biogas plant or other plants and procedures for the processing of biomass. The subdivision of this tank into a biomass-feeding chamber and a pump chamber achieves the subdivision through the arrangement of a drop wall, whereby a complete separation of both chambers is effected in a liquid-filled state. Both tank chambers are insulated and the pump chamber is preferably lowered relative to the feeding chamber by virtue of the tank floor being arranged aslant in a downward direction so that the two chambers can be separated gas-tight from each other. The supplied organic matter is directly fed in from the delivery vehicle, delivery equipment/device into the feeding chamber of the combi-tank. The biomass used is supplied by means of a pump by an in-feed of biologically-active fluid material, for example, re-circulating material from more advanced process stages of fermentation (e.g. from the fermenter), and it is added through a pipe system to the biomass having been fed in, whereby it is simultaneously mixed, suspended, mashed and hydrolysis is sustainably optimized.

Beyond hydrolysis, homogenization is achieved, which is supported by:
injected external fluid,
the substrate being mixed through with the tank contents from the pump chamber in the feeding chamber,
single or multiple conditioning of the material with the cutting pump in the pump chamber,
use of mechanical agitators,
optional use of ultrasound,
optional feed-in of the hydrolysis gas or other hydrogenous gases into the lower zones of the feeding chamber or the pump chamber that are filled with fluid.

In the pump chamber sump, extraneous materials are separated. Overall, the pump chamber serves fur the storing of further processable substrate, continuous feeding to the downstream plant according to need, and provision of a reservoir for hydrolysis bacteria.

The gas compartments of both chambers can be optionally separated on the gas side or, instead, he connected as needed, and the biogas quantities can be fed into the central gas grid of the biogas plant either by means of the natural gas pressure from the gas formation phase or can be returned to the lower fluid zone of the feeding chamber or, as needed, into the pump chamber by means of a ventilator. The dosing of the biomass mixture from the combi-tank in the subsequent process is carried out dependent on the present gas formation in the system by means of the volume flow-control. The tank opening in the ceiling of the mixing and combi-hydrolysis tank is closed outside of feeding times with an odour-inhibiting or gas-tight flap, and is filled, if necessary, through an injection shaft.

As an option for the further optimization of material decomposition, an ultrasonic module is integrated into the preparation and hydrolysis system. This ultrasonic module is suitable for the treatment of any fluids. In a special design variant, it can also be applied in combination with the mixing and combi-hydrolysis tank. According to the invention, a multi-stage, self-regulating ultrasonic disintegration system is provided, which is not installed between or externally in a separate tank, but which combines the required components and necessary elements in a compact design in one system for the direct attachment to, or installation in, the mixing and combi-hydrolysis tank, without requiring a separate building or container setup.

The ultrasonic module is comprised of the following elements:

system of piping, which can also be square or rectangular in some areas,

piping elements, piping shutoff elements, measuring instruments,

test connections with equipment for testing, measuring and backwashing,

sonotrodes and integrated reflectors, fluid transporting units, and

backwashing units, fixtures and passage or connection equipment,

at least one reversible pump with rotation speed control.

The ultrasonic module is installed, supported by support 28, on the wall 1 of the tank which is shown containing fermentation substrate 2. The ultrasonic module includes substrate lines 23, sliders 24, reversible pump 25 with rotation speed control, sonotrodes 26, and gauge connections/flushing nozzles 27.

The ultrasonic module transports the medium to be disintegrated from the mixing and combi-hydrolysis tank through pipe-like elements with an integrated transporting unit. It is mounted on or in the mixing and combi-hydrolysis tank. The fluid is transported on centrically integrated sonotrodes in the pipes via shutoff devices, piping elements, volume flow-measuring devices and devices for mounting sensors and measuring elements, as well as by the transporting unit, preferably in a vertical inflow.

The sonotrodes are coupled with matching reflectors, which are centrically arranged in the media flow at suitable spacing in parallel to the probe.

The system is designed so that the medium to be disintegrated is transported from the mixing and combi-hydrolysis tank to the disintegration probes.

The inflow takes place in a single or multi-stage process. In between the stages of disintegration, the effects from the individual disintegration nozzles can be assessed by means of integrated gauge connections and measuring elements. In addition, the viscosity and/or temperature, power consumption of the sonotrodes and the transporting unit can be measured. Depending on the measuring or analysis results, the system can activate further stages via the transporting unit (preferably a pump), whereby it is possible to increase the intensity (lower flow speed), reduce the intensity, or initiate backwashing.

The configuration in stages and the number of sonotrodes can be adjusted to the quantity and intensity of the disintegration. The integrated transporting units or devices can effect a counter-flow direction in order to, for example, perform backwashing. If necessary, the transporting unit can adjust the transporting unit capacity to needs/requirements (for example, rotation speed control).

The intake and flushing ports are secured against reciprocal effects by flow-guiding devices and, respectively, by spatial arrangement in the system.

The system is able to increase the effects and function by means of a system control unit based on the communication between the setting and closing elements, the transporting unit, measuring elements and related analysis elements, the volume-measuring instrument and the communication with any subordinate control or its own control.

It is even possible to install this system—with exception of the transporting unit—within the fluid tank. All aforementioned components and required elements are combined in one system fur direct attachment to, or installation in, the mixing and combi-hydrolysis tank. A separate building or container setup is not necessary.

By virtue of its design, this ultrasonic module is able to measure the effect from the sonication directly by means of the integrated control unit, as well as to modify and, if needed, adjust the intensity by means of the volume flow-control or flow direction change (different passage of the fluid to be treated over a different number of sonotrodes).

Also, the self-cleaning function of the system is enabled through the reversal or change of the flow direction; as well it is possible to increase the volume flow and increase the flow speed at a ratio up to 1:10, for example, which can be configured at regular intervals in the sonication system for prophylaxis.

The system can be equipped with all common retail sonotrodes for in-pipe or on-pipe installation (thus, the sonotrodes are integrated both directly in the volume flow of the fluid to be treated or on the exterior wall of the pipe or in the exterior wall of the pipe).

Surprisingly, it became apparent that the wave-like shape of the piping of the ultrasonic system ensures, on the one hand, that the system is hydraulically optimized and, on the other hand, that a compact structural shape is achieved in observation of the space requirement for all the components to be integrated. Through the variation of the number of “waves,” the system can contain different numbers of sonotrodes or sonication areas, and it can thus be designed or built for differing sonication outputs.

The system can be installed on or in the mixing and combi-hydrolysis tank, and also as a bypass system or inline system.

The advantages of combining the mixing and combi-hydrolysis tank with the ultrasonic module, according to the invention, are presented, for example, in that the investment costs for an ultrasonic module are lowered by approx. 50% compared to the present cost of about EURO 200 k. In addition, these systems also lower operating costs considerably as direct connection to the mixing and combi-hydrolysis tanks reduces the transport paths by many times and can also be installed in a way that is beneficial for the flow and safe from clogging.

Furthermore, the ultrasonic module does not require any building-like enclosure, and measures of insulation and protection against the weather, as for common pipe-work installations, are sufficient.

FIG. 2 shows a side view of the ultrasonic module (14). In this example, the ultrasonic module is mounted on the exterior side of the tank's inside wall. FIG. 3 shows a top view of the ultrasonic module. It can be seen that the ultrasonic treatment takes place directly in the substrate line—thus, no extra tank is necessary—and the sonotrodes are arranged either inside or outside the substrate lines. Likewise shown are the gauge connections or flushing nozzles and the sliders (piping shutoff elements), as well as the pump (transporting unit).

FIG. 3 also shows that a second stage can be connected to the ultrasonic treatment if necessary. The wave-like shape of the ultrasonic module, according to the invention, is shown particularly clearly in FIG. 3.

1. Apparatus for the processing of biomass, comprising:
an opening with the ceiling of the first chamber adjacent the first end wall for feeding of biomass into the first chamber whereby the first chamber comprises a biomass feeding chamber and a pump in the second chamber whereby the second chamber comprises a pump chamber; and
a tank having a floor including a slanted floor portion:
an interior first drop wall offset laterally from the slanted floor, and extending downwardly from a ceiling of the tank to above the tank floor to divide the tank into two wherein the first drop wall is configured so that lower end of the drop wall is below a lowest liquid level in the tank at all times, the two chambers which communicate for transport of liquid between the two chambers and between which the first drop wall prevents flow of gas above the liquid level between the two chamber, a first of the chambers having an end at a first end wall of the tank and a second of the chambers having an end at a second end wall of the tank;
an interior second drop wall extending downwardly form the tank ceiling proximate the opening and being configured so that lowest end is at all times below a lowest liquid level with tank and thus prevents transport of gas between an area of the tank opening and the rest of the tank.

2. Apparatus according to claim 1, further comprising an injection system installed in an upper area of the feeding chamber and which is configured for moistening of biomass in the biomass feeding chamber and additionally comprising at least one agitator in the feeding chamber, a flushing connection, at least one gas outlet, and a gas return line for an in-feed into at least one of the pump chamber and the feeding chamber.

3. Apparatus according to claim 2, further comprising an odour-inhibiting or gas-tight lockable closure for the opening.

4. Apparatus according to claim 3, wherein the pump is a cutting pump is installed in the pump chamber on a portion of the floor lower than the remaining tank floor and forming a pump sump above the liquid level.

5. Apparatus according to claim 4, further comprising conduits communicating with the tank and configured for recirculation of biomass substrate liquid and a multi-stage, self-regulating ultrasonic module for disintegration of recirculating substrate liquid.

6. Apparatus according to claim 5, wherein the ultrasonic module comprises the following elements:
a) at least one substrate line for recirculation of the biomass substrate liquid;
b) at least one transporting unit;
c) one or more sonotrodes;
d) device for the testing or measuring of liquid parameters of the recirculating substrate liquid; and
e) device for connection to the tank,
wherein the sonotrodes are arranged inside of the substrate line or on exterior surface of the substrate line.

7. Apparatus according to claim 6, comprising a plurality the substrate lines are arranged in a wave-like shape and wherein the transporting out comprises a reversible pump with rotation speed control.

8. Apparatus according to claim 7, wherein the ultrasonic module further comprises reflectors adherent the sonotrodes and further comprising a control unit configured to control the pump by gauged parameters.

9. Process for combined feeding, mixing, dosing, hydrolysis, disintegration and methane enrichment of biomass as well as for the improvement of viscosity for use in a biogas plant or other plant by means of the device according to claim 8, comprising the following steps:
a) feeding the biomass feeding chamber with biomass substrate including liquid through the opening directly from a delivery vehicle or delivery device, the delivery equipment/device, so that fresh substrate is added to substrate liquid contained in the tank subsequent to start-up of the process;
b) injecting into the tank through the injection system and from the pump chamber fresh substrates with biologically-active liquid from an external source or a fermenter of a plant treating biomass or with another liquid, whereby hydrolysis of the biomass begins and liquid level in the tank rises to an upper level;
c) with the agitator mixing and thereby beginning homogenization of the biomass;
d) transferring the biomass which has been mixed into the pump chamber and by means of the cutting pump comminuting and further homogenizing the biomass;
e) withdrawing from the tank gas formed by hydrolysis of the biomass and feeding into at least one of the pump chamber and the feeding chamber more biomass and, optionally, hydrogenous gases for stimulation of methane formation by supporting the living conditions of hydrogen-oxidizing archaea or other hydrogen-oxidizing microorganism in the tank; and
f) transferring of thereby processed biomass from the pump chamber into a fermenter or other digester.

10. The process according to claim 9, wherein transferring of the processed biomass from the controlled dependent upon gas formation in the tank, by means of volume flow-control.

11. The process according to claim 9, wherein the pump chamber serves as a reservoir for hydrolysis or hydrogen-oxidizing microorganism.

12. The process according to claim 9, wherein a biologically-active fluid substance from a more advanced process stage of fermentation is added to the biomass in the tank through the injection system by means of a pump external to the tank, whereby it is simultaneously mixed, suspended, mashed into the biomass and hydrolysis is sustainably optimized.

13. This process according to claim 9, wherein hydrogenous gases from an external source are fed into the tanks through a gas return line.

14. The process according to claim 12, further comprising ultrasonic treatment in the ultrasonic module of the biologically-active fluid substance from a more advanced process stage of fermentation mixed with the biomass, therein increasing decomposition by hydrolysis and circulating the mixture so that the mixture treated with ultrasound thereupon again undergoes hydrolysis.

15. The process according to claim 14, wherein
a) ultrasonic treatment takes place inside the substrate line, and
b) parameters of liquid in the tank are measured inside the substrate line at connections of gauges with the substrate line, and
c) the pump is controlled by means of measured parameters, whereby intensity of pumping is increased or reduced or backwashing is initiated.
